(19) United States
(12) Patent Application Publication
(10) Pub. No.: US 2026/0024207 A1
(43) Pub. Date: Jan. 22, 2026

(54) SYSTEMS AND METHODS FOR HARMONIZING MEDICAL IMAGE AND DATA AND PROVIDING OPERATION INSIGHTS USING AI

(71) Applicant: Subtle Medical, Inc., Menlo Park, CA (US)

(72) Inventors: Gregory Zaharchuk, Stanford, CA (US); Praveen Kumar Gulaka, Redwood City, CA (US); Britney Alexia Tyler, Cedar Hill, TX (US)

(21) Appl. No.: 19/342,850

(22) Filed: Sep. 29, 2025

Related U.S. Application Data
(63) Continuation of application No. PCT/US2024/021696, filed on Mar. 27, 2024.
(60) Provisional application No. 63/493,094, filed on Mar. 30, 2023.

(51) Int. Cl. G06T 7/00; G06T 7/20; G06V 10/764; G16H 10/60; G16H 30/20; G16H 30/40

(52) U.S. Cl. CPC G06T 7/0012; G06T 7/20; G06V 10/764; G16H 10/60; G16H 30/20; G16H 30/40; G06T 2200/24; G06T 2207/20081; G06T 2207/30168; G06V 2201/03

(57) ABSTRACT

Methods and systems are provided for computer-implemented method for providing data-driven insights. The method comprises: receiving input medical images of a subject; utilizing deep learning-based algorithm to determine a quality of the input medical images, standardize a format or name of the input medical images and/or assess a completeness of a protocol associated with acquiring the input medical images; and generating insights based at least in part on the quality of the input medical image pr the completeness of the protocol, and displaying the insights on a graphical user interface (GUI).

### CROSS-REFERENCE TO RELATED APPLICATION

This application is a continuation of International Application No. PCT/US2024/021696 filed on Mar. 27, 2024, which claims priority to U.S. Provisional Application No. 63/493,094 filed on Mar. 30, 2023, the content of which is incorporated herein in its entirety.

### BACKGROUND

Radiologists may utilize or compare sequential imaging studies acquired on different medical hardware systems e.g., magnetic resonance (MR) hardware systems. Even images are acquired using the same imaging modality, because each manufacturer's images show different contrast or distortions due to different design considerations, this task can be challenging. Clinical imaging trials can be more challenging if multiple vendor scanners are involved. Medical images may have different appearances, image qualities, image formatting, file format, names, etc. across different acquisition sessions, vendors or imaging systems that are used. Due to the various different image and data formats, phases, users often may need to use different downstream software to process the respective data. This may increase the burden on the users to know various different display protocols (e.g., a series of actions performed to arrange images for optimal softcopy viewing).

Further, imaging modality such as magnetic resonance imaging (MRI) has been used to visualize different soft tissue characteristics by varying the sequence parameters such as the echo time and repetition time. Through such variations, the same anatomical region can be visualized under different contrast conditions and the collection of such images of a single subject is known as multi-contrast MRI. Multi-contrast MRI provides complimentary information about the underlying structure as each contrast highlights different anatomy or pathology. For instance, complementary information from multiple contrast-weighted images such as T1-weighted (T1), T2-weighted (T2), proton density (PD), diffusion weighted (DWI) or Fluid Attenuation by Inversion Recovery (FLAIR) in magnetic resonance imaging (MRI) has been used in clinical practice for disease diagnosis, treatment planning as well as down-steam image analysis tasks such as tumor segmentation. Each contrast provides complementary information. However, due to scan time limitations, image corruptions due to motion and artifacts, and different acquisition protocols, one or more of the multiple contrasts may be missing, unavailable or unusable. This poses a major challenge for the radiologists and the automated image analysis pipelines.

### SUMMARY

The present disclosure addresses the above needs by providing systems and methods that are capable of automatically detecting image quality, adjusting image reformatting, standardizing and harmonizing data and naming (e.g., file name), and providing a dashboard for operation insights across all the data. Methods and systems herein may provide a platform that is capable of automatically detecting image quality, adjusting image reformatting, standardizing and harmonizing data and naming (e.g., file name), and providing a dashboard for operation insights across all the data.

In an aspect of the present disclosure, a system for medical image processing is provided. The system comprises a first component utilizing a deep learning-based algorithm to determine one or more quality metrics of input medical images of a subject, the one or more quality metrics comprise a motion score; a second component configured to transform an original description about the input medical images into a standardized description about the input medical images; a third component configured to process metadata of the input medical images to extract insights related to one or more workflow metrics; and a graphical user interface (GUI) coupled to the first component, the second component and the third component to display the standardized description about the input medical images, a recommendation to rescan at least one of the input medical images based at least in part on the one or more quality metrics generated by the first component and the insights extracted by the third component.

In some embodiments, the one or more quality metrics further comprise a signal-noise-ratio, and an extent coverage of a body part of the subject. In some cases, the extent coverage comprises an indication of an incompleteness of a protocol associated with acquiring the input medical images. In some instances, the recommendation to rescan comprises a guidance to rescan one or more of the input medical images with the protocol. In some cases, the extent coverage comprises an indication that a tissue of the subject is incomplete or invisible. In some instances, the recommendation to rescan comprises a guidance to rescan the tissue that is incomplete or invisible.

In some embodiments, the standardized description about the input medical images contains at least a tissue name or a contrast description that is inconsistent with the original description. In some cases, the tissue name is based at least in part on a tissue identified by a body part classification model of the system. In some cases, the contrast description is based at least in part on a contrast level predicted by a contract classification model of the system.

In some embodiments, the input medical images are processed by a transformer model to generate a synthesized image with a contrast missing from the input medical images. In some cases, the input medical images are processed by the transformer model prior to being processed by the first component. In some cases, the recommendation to rescan is generated based at least in part on the synthesized image.

In some embodiments, the one or more workflow metrics comprise a utilization of an imaging device that acquires the input medical images, and a technologist productivity. In some embodiments, the metadata is obtained from an DICOM metadata or an HL7 (Heath Level Seven) message.

In a related yet separate aspect, a method is provided for medical image processing. The method comprises: (a) processing input medical images of a subject utilizing a deep learning-based algorithm to determine one or more quality metrics, wherein the one or more quality metrics comprise a motion score; (b) transforming an original description about the input medical images into a standardized description about the input medical images; (c) processing metadata of the input medical images to extract insights related to one or more workflow metrics; and (d) providing a graphical user interface (GUI) to display the standardized description about the input medical images, a recommendation to rescan at least one of the input medical images based at least in part on the one or more quality metrics and the extracted insights.

In some embodiments, the one or more quality metrics further comprise a signal-noise-ratio, and an extent coverage of a body part of the subject. In some cases, the extent coverage comprises an indication of an incompleteness of a protocol associated with acquiring the input medical images. In some instances, the recommendation to rescan comprises a guidance to rescan one or more of the input medical images with the protocol. In some cases, the extent coverage comprises an indication that a tissue of the subject is incomplete or invisible. In some instances, the recommendation to rescan comprises a guidance to rescan the tissue that is incomplete or invisible.

In some embodiments, the standardized description about the input medical images contains at least a tissue name or a contrast description that is inconsistent with the original description. In some cases, the tissue name is based at least in part on a tissue identified by a body part classification model of the system. In some cases, the contrast description is based at least in part on a contrast level predicted by a contract classification model of the system.

In some embodiments, the method further comprises, prior to (a), processing the input medical images by a transformer model to generate a synthesized image with a contrast missing from the input medical images. In some cases, the recommendation to rescan is generated based at least in part on the synthesized image.

In some embodiments, the one or more workflow metrics comprise a utilization of an imaging device that acquires the input medical images, and a technologist productivity. In some embodiments, the metadata is obtained from an DICOM metadata or an HL7 message.

Additional aspects and advantages of the present disclosure will become readily apparent to those skilled in this art from the following detailed description, where only illustrative embodiments of the present disclosure are shown and described. As will be realized, the present disclosure is capable of other and different embodiments, and its several details are capable of modifications in various obvious respects, all without departing from the disclosure. Accordingly, the drawings and descriptions are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1A schematically illustrates a platform comprising a processing layer that is capable of processing various different input medical data.

FIG. 1B schematically illustrates various components of the platform.

FIG. 2 shows an example of generating standardized image series name.

FIG. 3A shows an example of generating standardized name for the input image or files (e.g., series naming) as well as identifying contrast; FIG. 3B shows examples of standardized series description or name for the input image or files (e.g., series naming) generated by a Meta Data Parser (MDP) component.

FIG. 4 shows an example of operational workflow efficiency or utilization.

FIGS. 5 and 6 show a platform links HL7 and DICOM (Digital Imaging and Communications in Medicine) to determine granular radiology workflow steps and determine the utilization metrics.

FIGS. 7-13 show examples of GUI for providing an insight dashboard.

FIG. 14 and FIG. 15 show examples of images with high and low quality metrics identified by the system herein.

FIG. 16 shows an example of series of images realigned by a reformat component of the system.

FIG. 17 shows an example of an insight dashboard.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

It is desirable to provide systems and methods that are capable of automatically detecting image quality, adjusting image reformatting, standardizing and harmonizing data and naming (e.g., file name), and providing a dashboard for operation insights across all the data. Methods and systems herein may provide a platform that is capable of automatically detecting image quality, adjusting image reformatting, standardizing and harmonizing data and naming (e.g., file name), and providing a dashboard for operation insights across all the data.

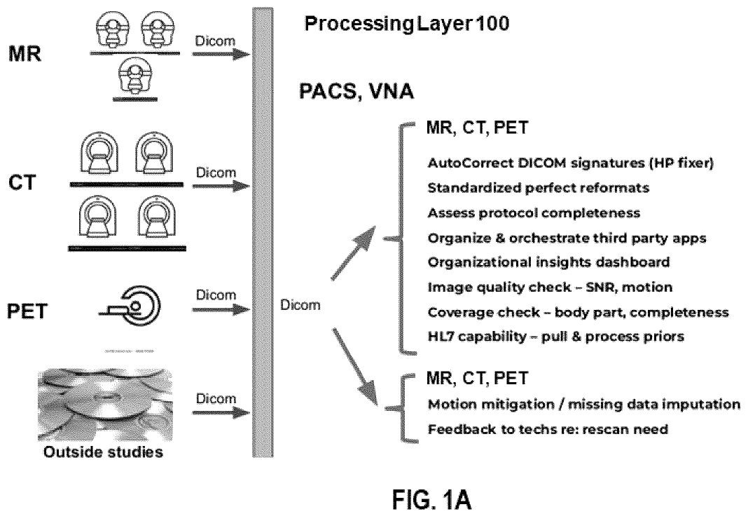

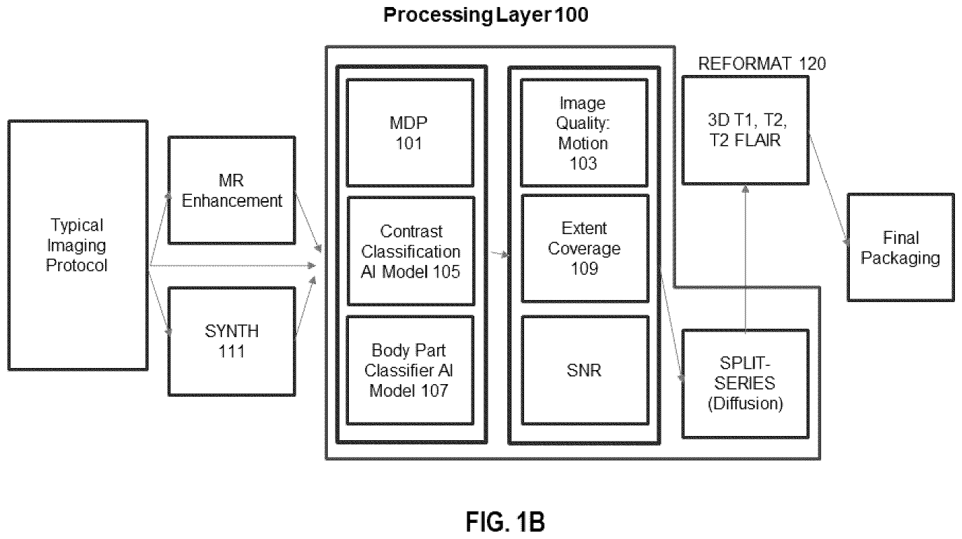

The platform may utilize deep learning-based algorithms for synthesizing medical images of a higher image quality, synthesizing a missing contrast, synthesizing images in a standardized format, and/or harmonizing data and name of a file. As illustrated in FIG. 1A, the platform herein may comprise a processing layer that is capable of processing various different input medical data. the input medical data may include medical images or data acquired using different modalities (e.g., MR, CT, PET), acquired using imaging systems provided by different vendors, the image files or data may be in inconsistent formats, quality, naming, etc. The processing layer may utilize deep learning-based algorithms to allow for auto-correcting file format (e.g., DICOM signatures such as HP fixer), standardizing image reformats, automatically detecting image quality (e.g., image quality check on SNR, motion) and/or performing coverage check (e.g., detect whether body part or tissue is completely visible), performing motion mitigation and missing data imputation and automatically generate feedback to users such as recommending rescan.

The processing layer may further be capable of assessing protocol completeness, organizing and orchestrating third party applications, providing HL7 capability (e.g., international standards used by healthcare providers transferring clinical and administrative data between software applications) to pull and process prior information and the like. The platform may also be capable of providing a graphical user interface (GUI) allowing users to access organizational insights via a dashboard.

FIG. 1B shows various components of the processing layer 100. As described above, the processing layer 100 may a Meta Data Parser (MDP) component 101 configured to transform the file metadata or file names into standardized name or format. In some embodiments, the MDP component 101 may utilize natural language processing (NLP) to transform a series description into a standard description format such as according to UCSF convention. In some embodiments, the Meta Data Parser (MDP) component 101 may generate the standardized series description based at least in part on the original series description and metadata about the image data (e.g., pixel data, time stamp, file type, imaging device, etc.). For example, the Meta Data Parser (MDP) component 101 may take as input the original series description and metadata and predict a standardized series description that may contain information that is not explicitly in the original series description (e.g., tissue that is scanned, contrast, etc.) or that is inconsistent with the original series description. For example, the original description is AX T1 DNE without the tissue information and the predicted standardized series description is Brain Ax T1_SE with the tissue automatically identified by the MDP component. In some cases, the Meta Data Parser (MDP) component 101 may be capable of identifying an incorrect original series description and/or missing information in the original series description (e.g., tissue is missing or incorrect, contrast is missing or incorrect, etc.) and generate a new series description that is both in standardized format and with the corrected description. For example, the Meta Data Parser (MDP) component 101 may identify the correct tissue in the image data based on the meta data and/or output from the body part classifier AI model 107 and generate the new series description with the correct tissue name. and/or the contrast.

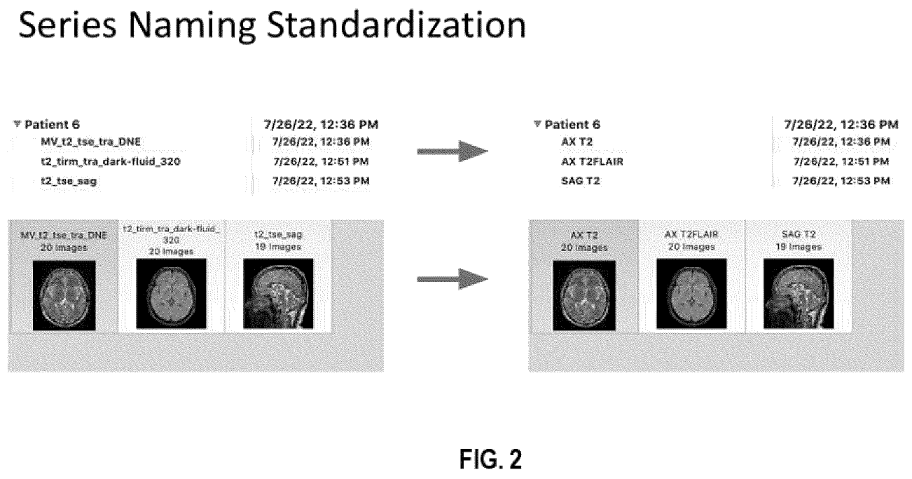

FIG. 2 shows an example of generating standardized image series name. As shown in the example, the name of the input image data may be updated by the platform and a standardized series name may be generated.

In another example, the Meta Data Parser (MDP) component 101 may identify the correct contrast in the image and generate the standardized series description with the correct contrast in the description. For example, the Meta Data Parser (MDP) component 101 may receive the contrast (e.g., no contrast agent administered) predicted by the contrast classification AI model 105 and detect that the original label of the contrast (e.g., post-contrast) in the image is incorrect. The Meta Data Parser (MDP) component 101 may generate the standardized series name with the correct description and metadata. The contrast classification AI model 105 may comprise a trained deep learning model that takes as input the image and metadata and outputs level of contrast agent administered to the patient when the image is acquired.

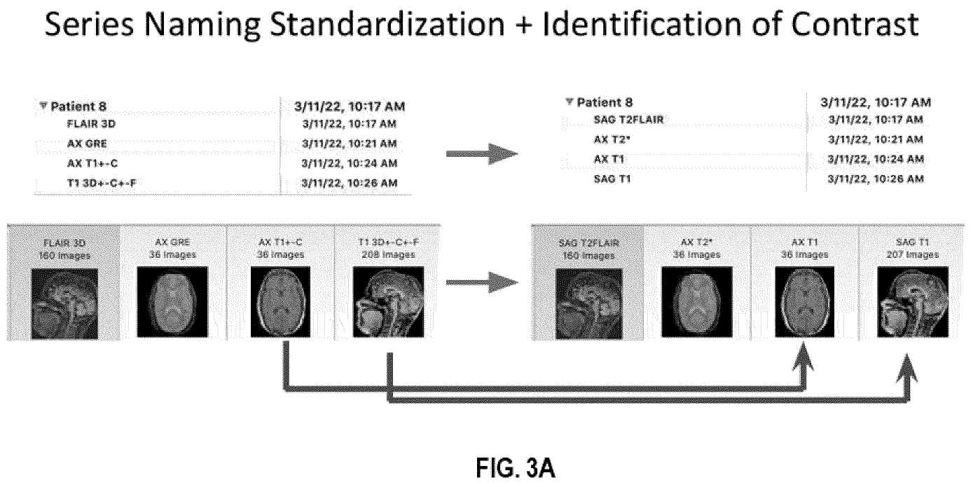

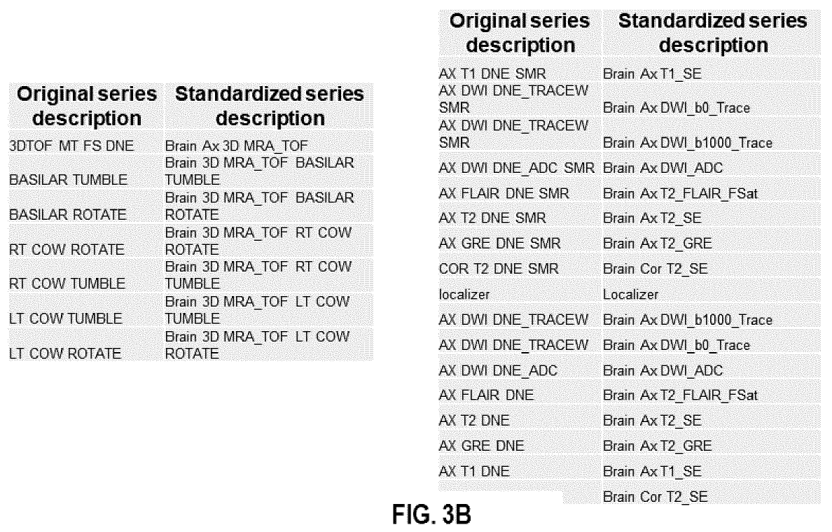

FIG. 3A shows an example of generating standardized name for the input image or files (e.g., series naming) as well as identifying contrast. The platform may be capable of checking the correctness of the input image such as whether the correct contrast is labeled. In the illustrated example, the input image AX T1 is labeled post-contrast and the platform may process the image and identify that the label is incorrect as no contrast is identified from the image. The platform may automatically correct the label or the name of the AX T1 image indicating no contrast.

FIG. 3B shows additional examples of standardized series description or name for the input image or files (e.g., series naming) generated by the MDP component 101. The MDP may receive the predicted image contrast from the contrast classification AI model 105 and/or the predicted tissue from the body part classifier AI model 107, and generates the standardized series description including the predicted image contrast and the predicted tissue.

Referring back to FIG. TA, the platform may comprise a DICOM (Digital Imaging and Communications in Medicine) node that receives images directly from scanners. The DICOM may analyze, fix inconsistent or incorrect formats, name, labels and the like in the input image, and/or route the images to PACS (Picture Archiving and Communication System) or VNA (vendor neutral archive). For instance, the platform may utilize AI or natural language processing (NLP) to fix naming of the data such that the naming is in a consistent format and the data is presented in a consistent order as described elsewhere herein.

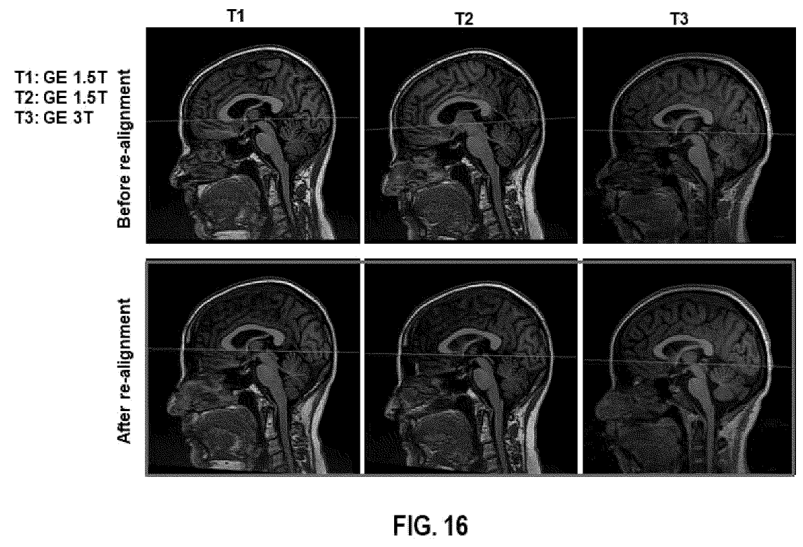

The platform may further allow for consistent hanging or display protocols regardless software upgrade, change or use of new scanners, new techs, repeat scans, etc. The platform may utilize AI to reformat the images from the correct source data with standard and consistent alignment. For example, the reformat component 120 in FIG. 1B may take the split series as input (e.g., 3D T1, T2, T2 FLAIR) and output reformatted series with consistent alignment. FIG. 16 shows an example of series of images realigned by the reformat component 120. The GUI may allow a user to manually align the split series by a user selected landmark. For instance, the user may click on a landmark in any of the series and the system may automatically align the series. The platform may orchestrate which series need to be sent to third parties (e.g., RAPID, other AI apps, etc.) and when to transmit the image series. For example, the images may be sent automatically without the need of human intervention.

The platform may utilize deep learning model to estimate of image quality, detect coverage in the image, and detect motion. As illustrated in FIG. 1B, the processing layer may comprise various quality control (QC) components to generate various QC metrics including, for example, image quality (IQ) component 103 for generating an IQ metric or motion metric, extent coverage component 109 for generating an extent coverage (EC) metric, and SNR (signal noise ratio) component for generating a SNR metric. For instance, the motion component 103 may comprise deep learning-based algorithms to detect a presence of motion in an image and/or quantify the motion by generating a score. The motion component 103 may determine whether the detected motion is acceptable or whether the image quality is acceptable. Upon determining the motion score fails a predetermined threshold, the motion component 103 may generate a recommendation for display to a user. For example, the recommendation may include a motion score, failure of the image quality and/or a recommendation for the user to rescan or retake an image.

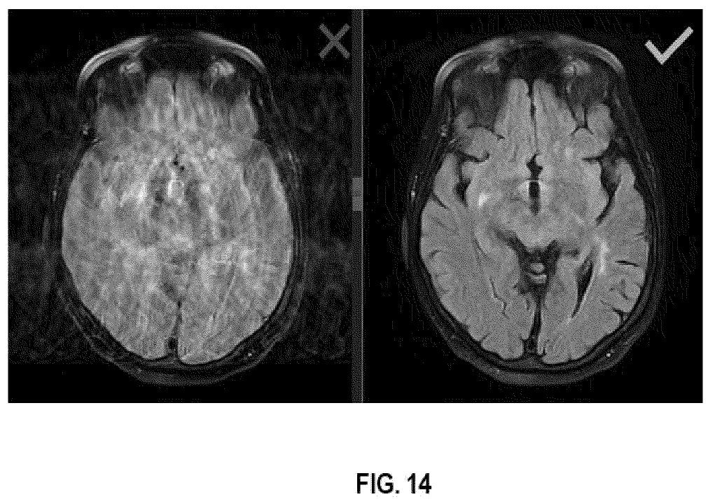

The system herein may generate recommendation to a user about the image quality and recommended action to mitigate the low quality. As shown in FIG. 14, the system may display which image failed or passed the image quality check. The failed image quality may be a combination of various quality metrics such as SNR (produced by the SNR component), motion score (generated by the motion component) and others. An image quality assessment component (e.g., SNR component, motion component, extent coverage component, etc.) of the system may determine that an image fails to pass a predetermined threshold by comparing the various metrics to various predetermined threshold. In some cases, the threshold may be adjustable by a user. In some cases, the threshold may be automatically determined by the system based on empirical data. In some cases, in addition to displaying that an image failed the quality check, recommendation such as a rescan may be provided. In some cases, the rescan recommendation may be generated based on the type of image artifacts (e.g., motion or SNR). For example, if the motion score is below a predetermined respective threshold, the recommendation may instruct the user to rescan with suggestions to mitigate motion of the patient (e.g., shorter scan time, instruct patient not to move, etc.).

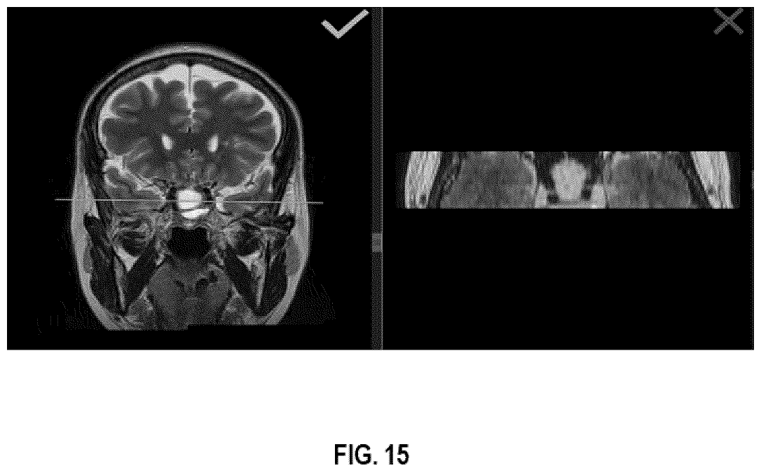

In some cases, the artifact may be a defect in coverage detected by the body part classifier AI model 107 and the extent coverage (EC) component. FIG. 15 shows an example of the image having a low EC metric (indicated by the cross mark) and good motion metric (indicated by the checkmark). The EC metric may be generated by the extent coverage (EC) component indicating whether a specific tissue or body part is incomplete or not visible. For example, upon performing a coverage check, the system may determine whether a body part or tissue is completely visible and if not, the system may generate a rescan recommendation with instruction or guidance to rescan a particular body part or adjust a position of the imaging device/patient. In some cases, the EC metric may indicate a completeness of a protocol associated with acquiring the input medical images. For example, the EC metric may indicate that a particular contrast image is missing or cannot be imputed by the synth component 111, and a recommendation to rescan may be generated to guide an acquisition of the medical image of the missing contrast. For instance, the recommendation to rescan is generated based at least in part on the synthesized image or the capability of the synth component to generate the synthesized image. The guidance may instruct the user to acquire an image with a contrast that cannot be imputed or to acquire an image that is required to generate an synthesized image by the synth component.

The platform may also be capable of detecting missing contrast, unavailable image and may recommend a rescan. Methods and algorithms for improving or standardizing image quality, format and detecting missing contrast can be the same as those described in U.S. patent Ser. No. 11/182,878 entitled “SYSTEMS AND METHODS FOR IMPROVING MAGNETIC RESONANCE IMAGING USING DEEP LEARNING”, U.S. Pat. No. 11,550,011 entitled “SYSTEMS AND METHODS FOR MAGNETIC RESONANCE IMAGING STANDARDIZATION USING DEEP LEARNING”, U.S. Publication US20230033442 entitled “SYSTEMS AND METHODS OF USING SELF-ATTENTION DEEP LEARNING FOR IMAGE ENHANCEMENT,” International Application PCT/US2022/048414 entitled “SYSTEMS AND METHODS FOR MULTI-CONTRAST MULTI-SCALE VISION TRANSFORMERS” which is incorporated by reference herein in its entirety. For example, multi-contrast MRI provides complimentary information about the underlying structure as each contrast highlights different anatomy or pathology. By varying the sequence parameters such as the echo time and repetition time, the same anatomical region can be visualized under different contrast conditions and the collection of such images of a single subject is known as multi-contrast MRI. For example, MRI can provide multiple contrast-weighted images using different pulse sequences and protocols (e.g., T1-weighted (T1), T2-weighted (T2), proton density (PD), diffusion weighted (DWI), Fluid Attenuation by Inversion Recovery (FLAIR) and the like in magnetic resonance imaging (MRI)). These different multiple contrast-weighted MR images may also be referred to as multi-contrast MR images. In some cases, one or more contrast-weighted images may be missing or not available. For example, in order to reduce scanning time, only selected contrasts are acquired while other contrasts are ignored. In another example, one or more of the multiple contrast images may have poor image quality that are not usable or lower quality due to reduced dose of contrast agent. The platform may be capable of detecting and/or synthesizing a missing contrast-weighted image based on other contrast images or to impute the missing data.

As illustrated in FIG. 1B, the missing data or missing contrast-weighted image may be generated by the Synth component 111. The synth component may comprise a Multi-contrast and Multi-scale vision Transformer (MMT) for predicting missing contrasts. The MMT may be trained to generate a sequence of missing contrasts based on a sequence of available contrasts. In some embodiments, the MMT based deep learning (DL) model may comprise a multi-contrast transformer encoder and a corresponding decoder that builds hierarchical representations of inputs and generates the outputs in a coarse-to-fine fashion. At test time or in the inference stage, the MMT model may take a learned target contrast query as input, and generate a final synthetic image as the output by reasoning about the relationship between the target contrasts and the input contrasts, and considering the local and global image context. For example, the MMT decoder may be trained to take a contrast query as an input and output the feature maps of the required (missing) contrast images.

The rescan recommendation may be produced after the missing data imputation. The MMT model may be capable of replacing lower quality contrasts with synthesized higher quality contrasts or imputing a missing contrast without the need for rescanning. However, in the case when the available contrasts as input to the MMT model is not sufficient to generate synthesized higher quality contrasts (e.g., due to completeness of a protocol associated with acquiring the input medical images), the processing layer 100 may generate a recommendation for rescan with instructions or guidance to acquire an image that is sufficient for the MMT model to synthesize a missing contrast. For instance, the guidance may include recommended position of the patient relative to the imaging device and/or the scanning parameters.

The platform may provide a graphical user interface (GUI) such as an insights dashboard to an entity (e.g., organization). The GUI may provide, for example, overview of imaging operations. The dashboard may provide, for example, how many scans are being done on each scanner, how long each is taking, how many rescans are being done, is image quality changing, and various other insight information. The platform may provide various other features or functions such as importing outside studies for conferences (e.g., tumor boards) and providing data mining at various level (e.g., organizational level).

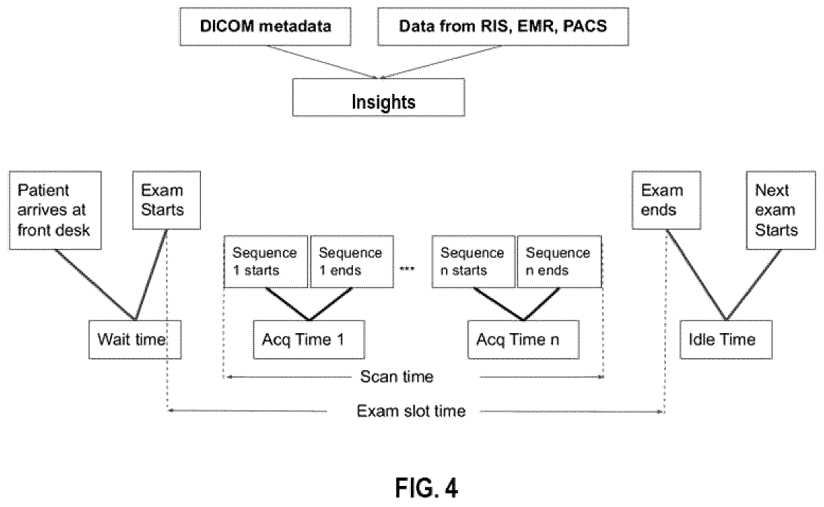

FIGS. 4-6 show examples of providing data driven insights. The platform may provide data-driven insights on operational assets such as Scanners, Techs, Radiologists and the like. In some cases, the system generated insights may include actionable recommendations. Users may be permitted to access, view and interact with the system generated insights via a GUI. Details and examples of the GUI are described later herein.

In some embodiments, the insights may comprise performance metrics. The performance metrices may comprise, for example, exam volume tracked across all sites and modalities of the customer, scanner utilization (e.g., scanner usage is monitored on hourly, daily or monthly granularity), exam type analysis (e.g., distribution of procedures across scanners and Technologists), tech productivity analysis (e.g., compare number of exams, type of exams/procedures and duration of running exams to identify training needs), Protocol Adherence across sites and scanners, Referring Physician (e.g., referrals is monitored by analyzing patterns in referrals and creating awareness), Backlog/Wait times (e.g., Exam ordered to completion of scan is monitored) and various others.

In some embodiments, the insights may comprise utilization metrics. Utilization metrics may be further utilized to optimize allotted appointment times to reflect average exam/scan times and the like. In some cases, the utilization metrics may provide insights about A: Gradient ON time from each sequence in an exam, B: Scan start, scan end from DICOMs, B-A: gives dead time during a scan, C: Appointment start, scan end from RIS, C-B+ 5 min for turn over: gives operational dead time, or analysis of imaging exams/procedures by scanner, technologist and referring physician.

In some embodiments, the insights may comprise quality metrics. As described above, the platform herein may be capable of detecting image quality, estimating motion, coverage in each series, determining protocol completeness (e.g., missing contrast or incorrect label of contrast) utilizing deep learning model. In some cases, the quality metrics provide by the platform may comprise image quality for each series, amount of motion for each series, extent coverage (e.g., which body part and completeness of the body part etc.) for each series, number of localizers per study, number of repeats (number of repeats where image quality is better on repeat), Protocol Completeness and others. In some cases, the platform may add to Technologist & Scanner data views to draw insights.

In some cases, the platform may build the insights utilizing HL7 message. Each HL7 message starts with a message header, corresponding to segment MSH, and defines the message's source, purpose, destination, and other syntax specifics like composite delimiters. MSH field 9, denoted by MSH-9, is particularly important since this specifies the type of message that is being transmitted (such as ADT, ORM??, “OMI”, ORU, ACK and so on. The segments present in a given message vary depending on the type of message that is being transmitted e.g., ORC, OBR (observation request), OBX (observation report).

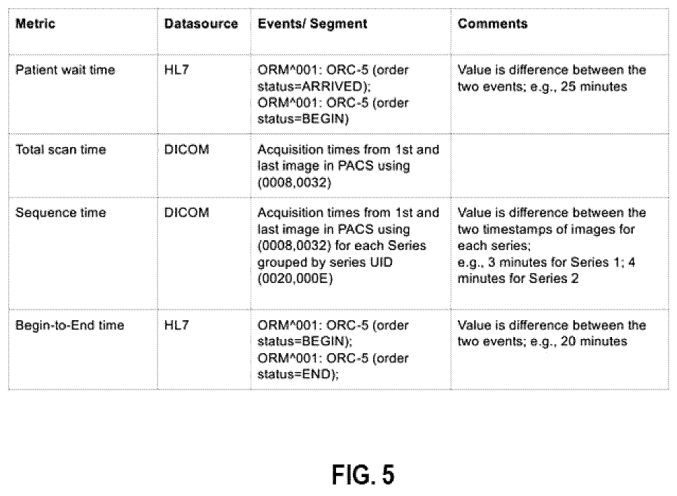

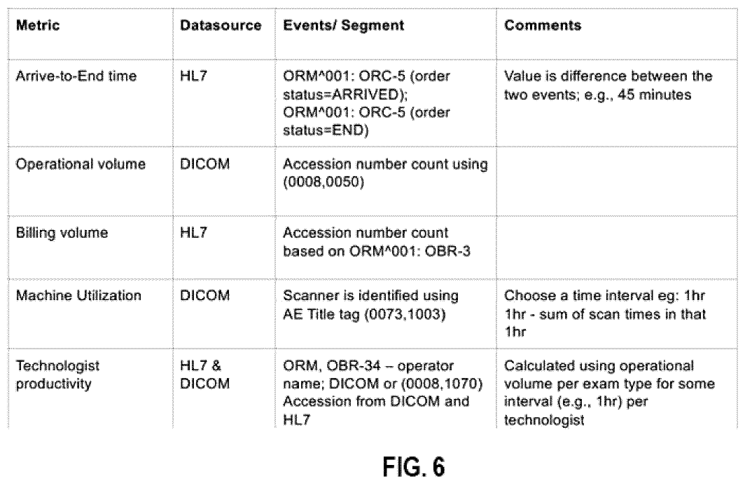

FIG. 4 shows an example of operational workflow efficiency or utilization. Various insights related to the operational workflow are extracted. As illustrated in FIGS. 5 and 6, the platform may link sources such as HL7 and DICOM of the imaging device to determine granular radiology workflow steps and determine the utilization metrics. As described above, the segment MSH field of the HL7 messages may be processed to extract the various workflow metrics including, but not limited to, patient wait time, begin-to-end time, arrive-to-end time, billing volume, technologist productivity and the like. The DICOM metadata may be processed to extract the metrics including, for example, total scan time, sequence time, operational volume, machine utilization, and the like.

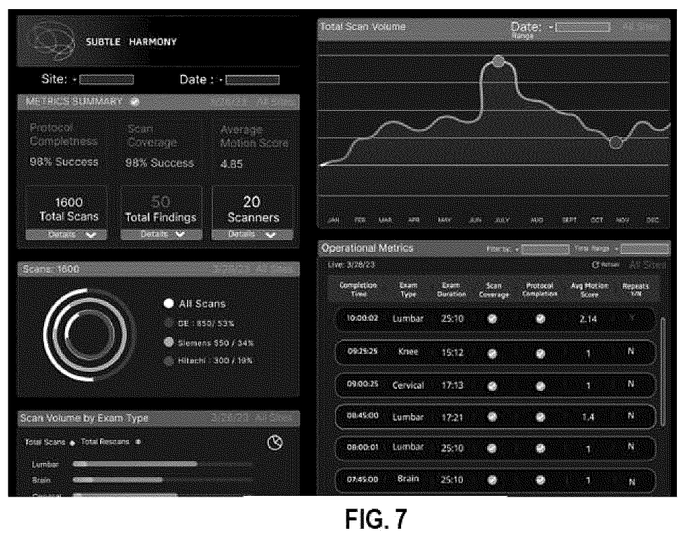

FIGS. 7-13 and FIG. 17 show examples of GUI for providing an insight dashboard.

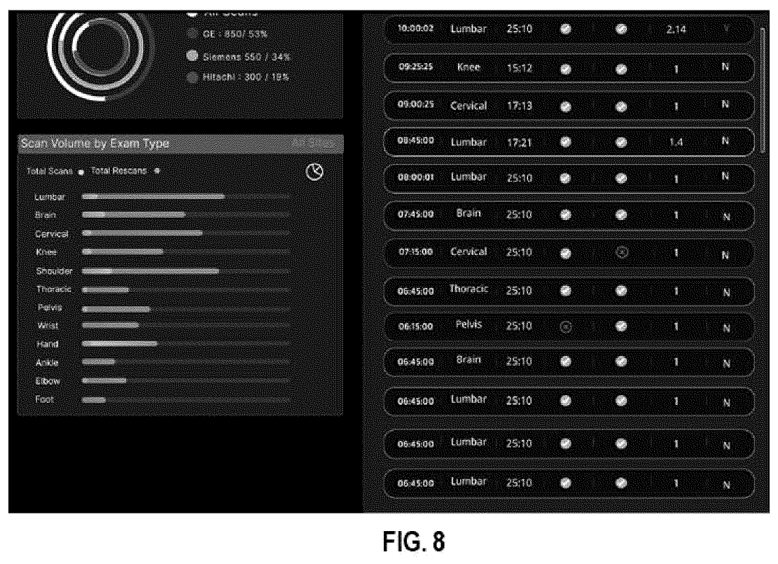

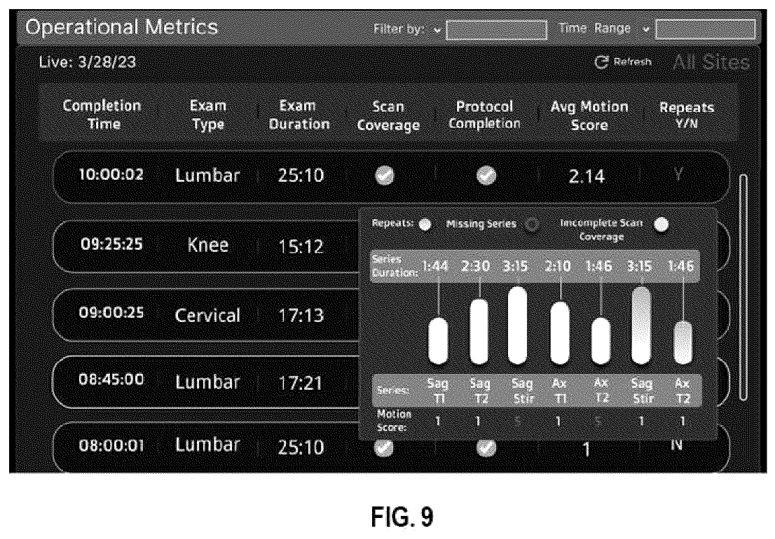

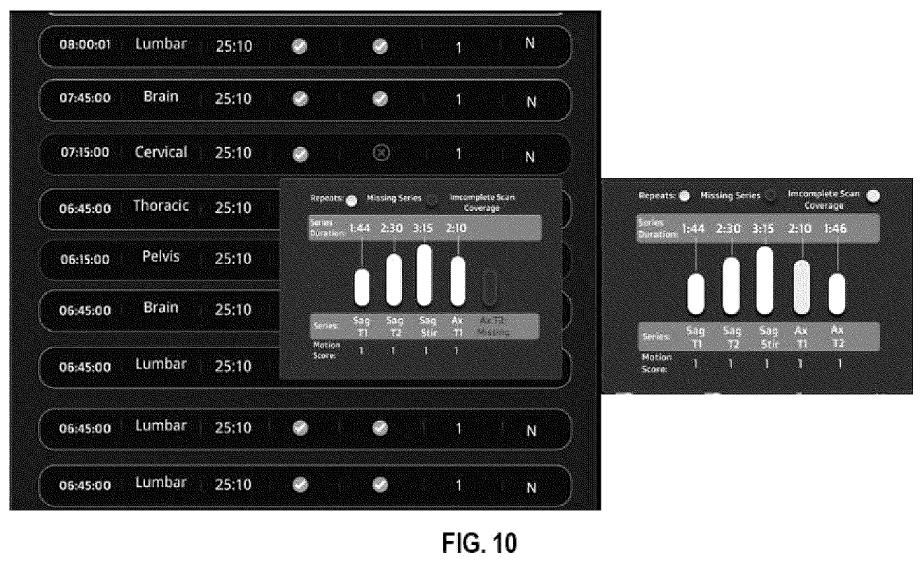

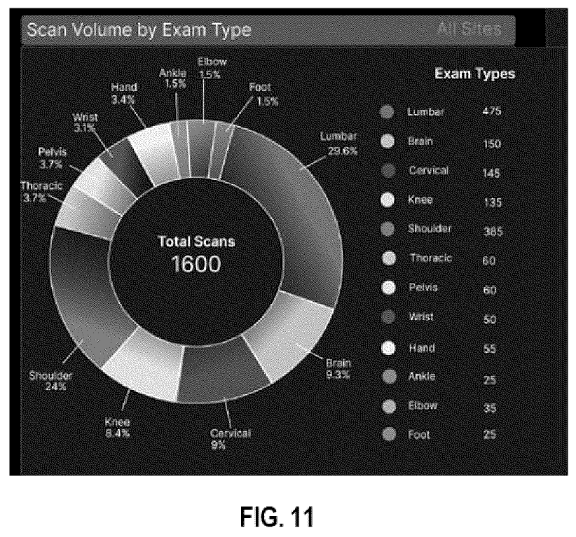

As shown in FIG. 7 and FIG. 8, the GUI may dynamically display information such as operational metrics, utilization metrics and quality metrics. The dashboard may provide a metrics summary displaying total scans, total findings, scanners, percentage of protocol completeness, scan coverage, average motion score and various other insights. The GUI may display scan volume by exam type (e.g., lumbar, brain, cervical, knee, shoulder, etc.). FIG. 9 and FIG. 10 show examples of GUI displaying operation metrics. The insights displayed on the dashboard may include, for example, number of repeats, missing series (e.g., Ax Ts: missing), incomplete scan coverage (e.g., Ax T1 incomplete coverage), motion score, and the like. The GUI may allow users to view the metrics in different formats such as bar plot or pie chart as shown in FIG. 11.

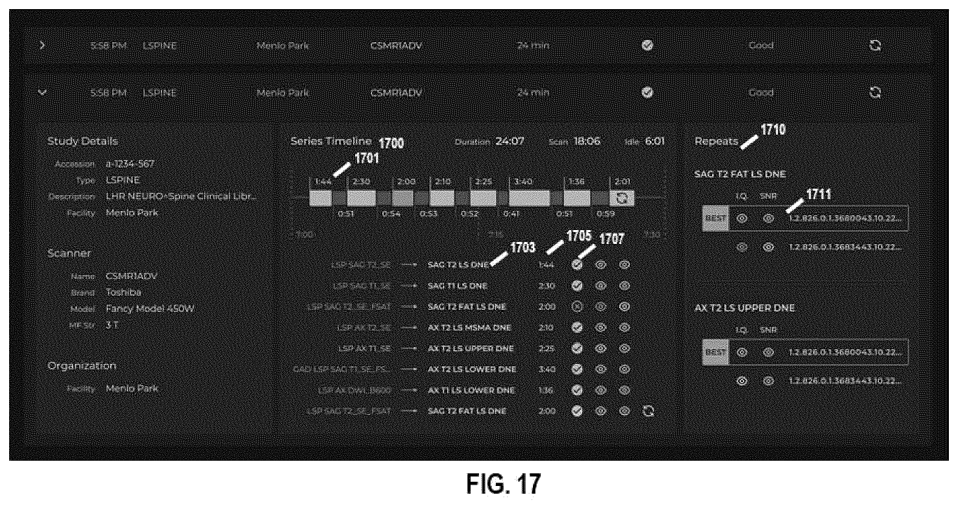

FIG. 17 shows another example of the dashboard displaying the key metrics. For example, the GUI may display repeats/rescan panel 1710 with IQ metric, SNR metric 1711 per series. The repeats panel 1710 may display the scan with the best quality control metrics 1711. The GUI may also display a series timeline 1700 automatically generated by the system. As shown in the example GUI, a series timeline 1700 may include a sequence of indicators 1701 representing a sequence of scans at different points of a timeline. The series timeline may be generated based one the workflow metrics (e.g., machine utilization, productivity, duration, begin-to-end time). Each point may include a scan duration 1705, a graphical indicator indicating whether the scan fails or passes the quality check 1707, and a standardized series description 1703 generated by the system.

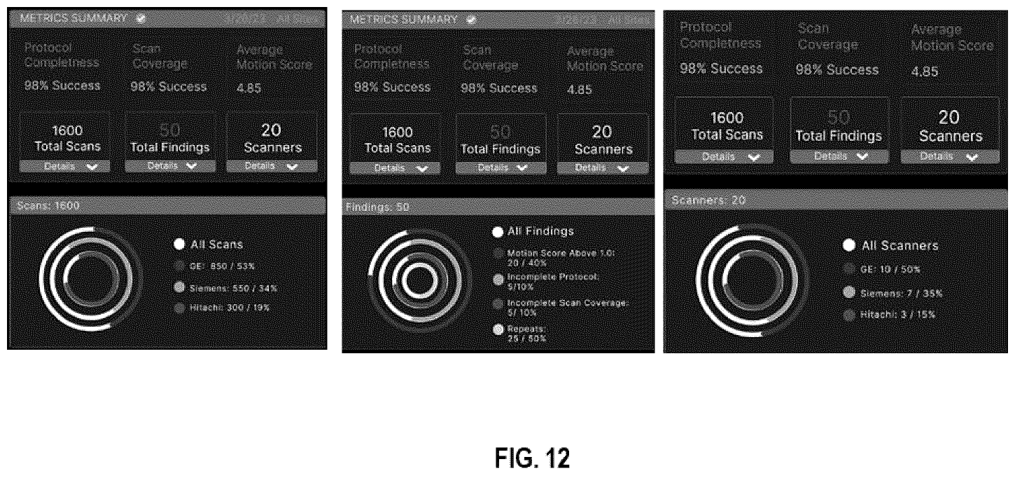

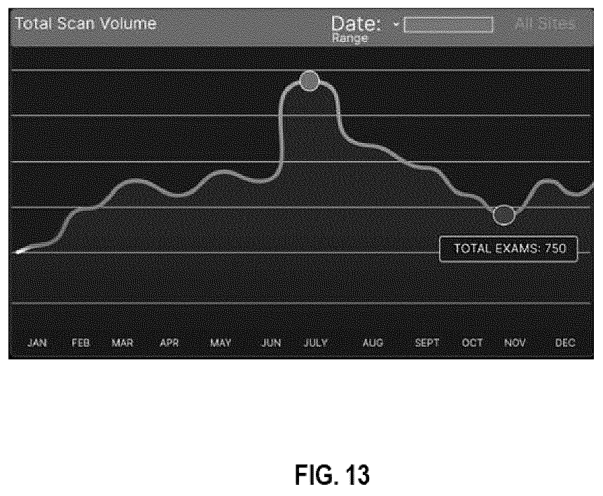

FIG. 12 shows an example of GUI providing insights on total scans (e.g., grouped by venders), total findings (e.g., motion score above a value, incomplete protocol percentage, incomplete scan coverage percentage, repeats percentage, etc.), and scanners (e.g., scanners by vendor). The GUI may be interactive. FIG. 13 shows an example of GUI allowing a user to access additional details such as by hovering cursor at any point on a total scan volume chart.

In some embodiments, the various functions and visual features may be provided virtually without the need to install, configure or manage any software. The data-driven operation workflow platform may be implemented on a cloud platform system (e.g., including a server or serverless) that is in communication with one or more user systems/devices via a network. The cloud platform system may be configured to provide the aforementioned functionalities to the users via one or more user interface or graphical user interfaces (GUIs), which may include, without limitation, web-based GUIs, client-side GUIs, or any other GUI as described above. For example, a user may access the coding challenge via a web-based-GUIs or within a web browser. In some cases, the graphical user interface (GUI) or user interface may be provided on a display. The display may or may not be a touchscreen. The display may be a light-emitting diode (LED) screen, organic light-emitting diode (OLED) screen, liquid crystal display (LCD) screen, plasma screen, or any other type of screen. The display may be configured to show a user interface (UI) or a graphical user interface (GUI) rendered through an application (e.g., via an application programming interface (API) executed on the user device or the user system, or on the cloud).

The platform may comprise computer systems and database systems, which may interact with the system. The computer system may comprise a laptop computer, a desktop computer, a central server, distributed computing system, etc. The processor may be a hardware processor such as a central processing unit (CPU), a graphic processing unit (GPU), a general-purpose processing unit, which can be a single core or multi core processor, or a plurality of processors for parallel processing. The processor can be any suitable integrated circuits, such as computing platforms or microprocessors, logic devices and the like. Although the disclosure is described with reference to a processor, other types of integrated circuits and logic devices are also applicable. The processors or machines may not be limited by the data operation capabilities. The processors or machines may perform 512 bit, 256 bit, 128 bit, 64 bit, 32 bit, or 16 bit data operations.

The computer system can communicate with one or more remote computer systems through the network. For instance, the computer system can communicate with a remote computer system of a user or a participating platform (e.g., operator). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system or the system via the network.

In some embodiments, one or more systems or components of the present disclosure are implemented as a containerized application (e.g., application container or service containers). The application container provides tooling for applications and batch processing such as web servers with Python or Ruby, JVMs, or Hadoop or HPC tooling. The various functions performed by the platform such as deep-learning based functions or generating insights and the like may be implemented in software, hardware, firmware, embedded hardware, standalone hardware, application specific-hardware, or any combination of these. The system, and techniques described herein may be realized in digital electronic circuitry, integrated circuitry, specially designed ASICs (application specific integrated circuits), computer hardware, firmware, software, and/or combinations thereof. These systems, devices, and techniques may include implementation in one or more computer programs that are executable and/or interpretable on a programmable system including at least one programmable processor, which may be special or general purpose, graphics processing unit (GPU), coupled to receive data and instructions from, and to transmit data and instructions to, a storage system, at least one input device, and at least one output device. These computer programs (also known as programs, software, software applications, or code) may include machine instructions for a programmable processor, and may be implemented in a high-level procedural and/or object-oriented programming language, and/or in assembly/machine language. As used herein, the terms “machine-readable medium” and “computer-readable medium” refer to any computer program product, apparatus, and/or device (such as magnetic discs, optical disks, memory, or Programmable Logic Devices (PLDs)) used to provide machine instructions and/or data to a programmable processor.

The network may establish connections among the components in the imaging platform and a connection of the imaging system to external systems. The network may comprise any combination of local area and/or wide area networks using both wireless and/or wired communication systems. For example, the network may include the Internet, as well as mobile telephone networks. In one embodiment, the network uses standard communications technologies and/or protocols. Hence, the network may include links using technologies such as Ethernet, 802.11, worldwide interoperability for microwave access (WiMAX), 2G/3G/4G/5G mobile communications protocols, asynchronous transfer mode (ATM), InfiniBand, PCI Express Advanced Switching, etc. Other networking protocols used on the network 1330 can include multiprotocol label switching (MPLS), the transmission control protocol/Internet protocol (TCP/IP), the User Datagram Protocol (UDP), the hypertext transport protocol (HTTP), the simple mail transfer protocol (SMTP), the file transfer protocol (FTP), and the like. The data exchanged over the network can be represented using technologies and/or formats including image data in binary form (e.g., Portable Networks Graphics (PNG)), the hypertext markup language (HTML), the extensible markup language (XML), etc. In addition, all or some of links can be encrypted using conventional encryption technologies such as secure sockets layers (SSL), transport layer security (TLS), Internet Protocol security (IPsec), etc. In another embodiment, the entities on the network can use custom and/or dedicated data communications technologies instead of, or in addition to, the ones described above.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

As used herein A and/or B encompasses one or more of A or B, and combinations thereof such as A and B. It will be understood that although the terms “first,” “second,” “third” etc. are used herein to describe various elements, components, regions and/or sections, these elements, components, regions and/or sections should not be limited by these terms. These terms are merely used to distinguish one element, component, region or section from another element, component, region or section. Thus, a first element, component, region or section discussed herein could be termed a second element, component, region or section without departing from the teachings of the present invention.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” or “includes” and/or “including,” when used in this specification, specify the presence of stated features, regions, integers, steps, operations, elements and/or components, but do not preclude the presence or addition of one or more other features, regions, integers, steps, operations, elements, components and/or groups thereof.

Reference throughout this specification to “some embodiments,” or “an embodiment,” means that a particular feature, structure, or characteristic described in connection with the embodiment is included in at least one embodiment. Thus, the appearances of the phrase “in some embodiment,” or “in an embodiment,” in various places throughout this specification are not necessarily all referring to the same embodiment. Furthermore, the particular features, structures, or characteristics may be combined in any suitable manner in one or more embodiments.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

What is claimed is:

1. A system for medical image processing comprising:
(a) a first component utilizing a deep learning-based algorithm to determine one or more quality metrics of input medical images of a subject, wherein the one or more quality metrics comprise a motion score;
(b) a second component configured to transform an original description about the input medical images into a standardized description about the input medical images;
(c) a third component configured to process metadata of the input medical images to extract insights related to one or more workflow metrics; and
(d) a graphical user interface (GUI) coupled to the first component, the second component and the third component to display the standardized description about the input medical images, a recommendation to rescan at least one of the input medical images based at least in part on the one or more quality metrics generated by the first component and the insights extracted by the third component.

2. The system of claim 1, wherein the one or more quality metrics further comprise a signal-noise-ratio, and an extent coverage of a body part of the subject.

3. The system of claim 2, wherein the extent coverage comprises an indication of an incompleteness of a protocol associated with acquiring the input medical images.

4. The system of claim 3, wherein the recommendation to rescan comprises a guidance to rescan one or more of the input medical images with the protocol.

5. The system of claim 2, wherein the extent coverage comprises an indication that a tissue of the subject is incomplete or invisible.

6. The system of claim 5, wherein the recommendation to rescan comprises a guidance to rescan the tissue that is incomplete or invisible.

7. The system of claim 1, wherein the standardized description about the input medical images contains at least a tissue name or a contrast description that is inconsistent with the original description.

8. The system of claim 7, wherein the tissue name is based at least in part on a tissue identified by a body part classification model of the system.

9. The system of claim 7, wherein the contrast description is based at least in part on a contrast level predicted by a contract classification model of the system.

10. The system of claim 1, wherein the input medical images are processed by a transformer model to generate a synthesized image with a contrast missing from the input medical images.

11. The system of claim 10, wherein the input medical images are processed by the transformer model prior to being processed by the first component.

12. The system of claim 11, wherein the recommendation to rescan is generated based at least in part on the synthesized image.

13. The system of claim 1, wherein the one or more workflow metrics comprise a utilization of an imaging device that acquires the input medical images, and a technologist productivity.

14. The system of claim 1, wherein the metadata is obtained from an Digital Imaging and Communications in Medicine (DICOM) metadata or an HL7 message.

15. A method for medical image processing comprising:
(a) processing input medical images of a subject utilizing a deep learning-based algorithm to determine one or more quality metrics, wherein the one or more quality metrics comprise a motion score;
(b) transforming an original description about the input medical images into a standardized description about the input medical images;
(c) processing metadata of the input medical images to extract insights related to one or more workflow metrics; and
(d) providing a graphical user interface (GUI) to display the standardized description about the input medical images, a recommendation to rescan at least one of the input medical images based at least in part on the one or more quality metrics and the extracted insights.

16. The method of claim 15, wherein the one or more quality metrics further comprise a signal-noise-ratio, and an extent coverage of a body part of the subject.

17. The method of claim 16, wherein the extent coverage comprises an indication of an incompleteness of a protocol associated with acquiring the input medical images.

18. The method of claim 17, wherein the recommendation to rescan comprises a guidance to rescan one or more of the input medical images with the protocol.

19. The method of claim 16, wherein the extent coverage comprises an indication that a tissue of the subject is incomplete or invisible.

20. The method of claim 19, wherein the recommendation to rescan comprises a guidance to rescan the tissue that is incomplete or invisible.
